(19) United States
(12) Patent Application Publication
(10) Pub. No.: US 2011/0250531 A1
(43) Pub. Date: Oct. 13, 2011

(54) Photosensitive Resin Composition for Color Filter, and Color Filter Using the Same

(75) Inventors: Ji-Yun KWON, Uiwang-si (KR); In-Jae LEE, Uiwang-si (KR); Ju-Ho JUNG, Uiwang-si (KR); Myung-Hwan CHANG, Uiwang-si (KR); Dong-Won SONG, Uiwang-si (KR); Han-Chul HWANG, Uiwang-si (KR); Sung-Hoon PARK, Uiwang-si (KR)

(73) Assignee: CHEIL INDUSTRIES INC., Gumi-si (KR)

(21) Appl. No.: 12/972,858

(22) Filed: Dec. 20, 2010

(30) Foreign Application Priority Data
Apr. 7, 2010 (KR) 10-2010-0031963

(51) Int. Cl. G03F 1/00; C07C 255/32

(52) U.S. Cl. 430/7; 558/401

(57) ABSTRACT

The present invention provides a photosensitive resin composition for a color filter including a methine-based dye represented by the following Chemical Formula 1, and a color filter fabricated using the same.

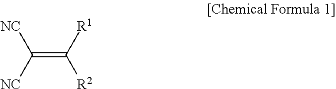

In Chemical Formula 1, R¹ and R² are respectively the same as defined in the specification.

### CROSS REFERENCE TO RELATED APPLICATIONS

This application claims priority to and the benefit of Korean Patent Application No. 10-2010-0031963 filed in the Korean Intellectual Property Office on Apr. 7, 2010, the entire disclosure of which is incorporated herein by reference.

### FIELD OF THE INVENTION

This disclosure relates to a photosensitive resin composition for a color filter and a color filter using the same.

### BACKGROUND OF THE INVENTION

Recently, the use of large screen liquid crystal displays (LCDs) has significantly increased, and thus there is a need to improve the performance of the same. The color filter is an important component of LCDs for providing colors among the many parts of a liquid crystal display. Accordingly, there is active research directed to improving process margins associated with the production of color filters. In addition, in order to increase color purity of a large screen LCD, a color filter can be fabricated using a photosensitive resin composition with an increased concentration of a colorant. Accordingly, there is need for a photosensitive resin composition that can be developed more quickly to increase productivity and yield in the manufacturing process and that has excellent sensitivity despite little exposure to light.

A photosensitive resin composition can be used to fabricate a color filter through various methods, such as dyeing, electrophoretic deposition (EPD), printing, pigment dispersion, and the like, in which three or more colors are coated on a transparent substrate. Recently, the pigment dispersion method has been more actively adopted.

However, the pigment dispersion method can result in decreased transmittance when, for example, a red pigment is included in larger amounts to form a red pixel displaying in a region with a high coloring property (a heavily-doped region) on a color coordinate. A yellow pigment can also be used in a high concentration to make a color coordinate out of a yellow axis and to provide high transmittance. However, when the amount of yellow pigment is increased, there can be problems associated with the resultant overall increased amount of pigment. Accordingly, the pigment dispersion method may not provide the desired level of color characteristics such as luminance, contrast ratio, and the like.

### SUMMARY

An exemplary embodiment of the present invention provides a photosensitive resin composition for a color filter that can have high luminance and contrast ratio.

Another embodiment provides a color filter fabricated using the photosensitive resin composition.

According to one embodiment, provided is a photosensitive resin composition for a color filter including a methine-based dye represented by the following Chemical Formula 1.

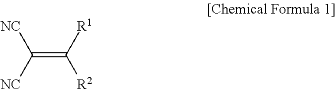

In Chemical Formula 1,

R¹ and R² are the same or different and are independently hydrogen, halogen, hydroxy, ether, amine, amine derivative, substituted or unsubstituted C1 to C20 alkoxy, substituted or unsubstituted C1 to C20 alkyl, substituted or unsubstituted C2 to C20 alkenyl, substituted or unsubstituted C2 to C20 alkynyl, substituted or unsubstituted C3 to C20 cycloalkyl, substituted or unsubstituted C3 to C20 cycloalkenyl, substituted or unsubstituted C3 to C20 cycloalkynyl, substituted or unsubstituted C2 to C20 heterocycloalkyl, substituted or unsubstituted C2 to C20 heterocycloalkenyl, substituted or unsubstituted C2 to C20 heterocycloalkynyl, substituted or unsubstituted C6 to C30 aryl, or substituted or unsubstituted C6 to C30 aryloxy.

The dye may have a solubility ranging from 3 to 20 in an organic solvent. The dye may have a maximum absorption wavelength in a wavelength region ranging from 400 to 460 nm and a transmittance ranging from 95 to 100% in a wavelength region ranging from 500 to 800 nm.

The dye may be thermally decomposed at a temperature ranging from 200 to 400° C., and may be included in an amount ranging from 0.1 to 20 wt % based on the entire amount of the photosensitive resin composition for a color filter.

The photosensitive resin composition for a color filter may further include a pigment. The pigment may be red or green.

The dye and the pigment may be mixed in a weight ratio ranging from 1:9 to 9:1.

The photosensitive resin composition for a color filter may further include an acrylic-based binder resin, a photopolymerization initiator, a photopolymerization monomer, and a solvent. In exemplary embodiments, the photosensitive resin composition for a color filter may include (A) the methine-based dye represented by the above Chemical Formula 1 in an amount ranging from 0.1 to 20 wt %; (B) the acrylic-based binder resin in an amount ranging from 1 to 30 wt %; (C) the photopolymerization initiator in an amount ranging from 0.1 to 10 wt %; (D) the photopolymerization monomer in an amount ranging from 1 to 15 wt %; and (E) the solvent in a balance amount.

The photosensitive resin composition for a color filter may further include a surfactant.

According to another embodiment, provided is a color filter fabricated using the photosensitive resin composition for a color filter.

Further embodiments of this disclosure are described in the following detailed description.

The photosensitive resin composition for a color filter can have excellent heat resistance and durability, high absorption in a wavelength region ranging from 400 to 460 nm, high luminance, and high contrast ratio, and thus can be used to provide a color filter that can have improved pattern characteristics, development processibility, chemical resistance, color reproducibility, and the like.

### BRIEF DESCRIPTION OF THE DRAWING

FIG. 1 is a spectral absorbance graph showing methine-based dyes (A-1) according to Examples 1 to 6 and (A-2) according to Examples 7 to 10.

### DETAILED DESCRIPTION

The present invention now will be described more fully hereinafter in the following detailed description of the invention and with reference to the accompanying drawings, in which some, but not all embodiments of the invention are described. Indeed, this invention may be embodied in many different forms and should not be construed as limited to the embodiments set forth herein; rather, these embodiments are provided so that this disclosure will satisfy applicable legal requirements.

As used herein, when a specific definition is not otherwise provided, the term “substituted” refers to one substituted with at least one substituent comprising halogen (F, Cl, Br, I), hydroxyl, C1 to C20 alkoxy, a nitro, cyano, amine, azido, amidino, hydrazino, hydrazono, carbonyl, carbamyl, thiol, ester, carboxyl or a salt thereof, sulfonic acid or a salt thereof, phosphoric acid or a salt thereof, C1 to C20 alkyl, C2 to C20 alkenyl, C2 to C20 alkynyl, C6 to C30 aryl, C3 to C20 cycloalkyl, C3 to C20 cycloalkenyl, C3 to C20 cycloalkynyl, C2 to C20 heterocycloalkyl, C2 to C20 heterocycloalkenyl, C2 to C20 heterocycloalkynyl, or a combination thereof in place of at least one hydrogen.

As used herein, when a specific definition is not otherwise provided, the terms “aniline derivative” and “amine derivative,” respectively, indicate that at least one hydrogen atom in NH₂ existing in aniline and amine is replaced with halogen, hydroxyl, carboxyl, ether, sulfonate, sulfonic acid, sulfoneamide, substituted or unsubstituted C1 to C20 alkyl, substituted or unsubstituted C1 to C20 alkoxy, or substituted or unsubstituted C6 to C30 aryl.

As used herein, when a specific definition is not otherwise provided, the term “ether group” refers to ROR′, wherein R and R′ are the same or different, and are independently substituted or unsubstituted C1 to C20 alkyl, substituted or unsubstituted C3 to C20 cycloalkyl, or substituted or unsubstituted C6 to C30 aryl.

As used herein, when a specific definition is not otherwise provided, the terms “heterocycloalkyl”, “heterocycloalkenyl”, “heterocycloalkynyl”, and “heterocycloalkylene”, respectively, refer to a cyclic group of a cycloalkyl, cycloalkenyl, cycloalkynyl, and cycloalkylene including at least one heteroatom of N, O, S, or P, or a combination thereof.

As used herein, when a specific definition is not otherwise provided, the term “(meth)acrylate” refers to both “acrylate” and “methacrylate”.

According to one embodiment, a photosensitive resin composition for a color filter includes a methine-based dye represented by the following Chemical Formula 1, and in exemplary embodiments includes (A) a methine-based dye represented by the following Chemical Formula 1, (B) an acrylic-based binder resin, (C) a photopolymerization initiator, (D) a photopolymerization monomer, and (E) a solvent.

(A) Dye

The dye may be yellow-based, and may be a methine-based dye represented by the following Chemical Formula 1.

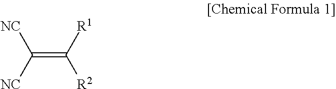

In Chemical Formula 1,

R¹ and R² are the same or different and may independently include hydrogen, halogen, hydroxy, ether, amine, amine derivative, substituted or unsubstituted C1 to C20 alkoxy, substituted or unsubstituted C1 to C20 alkyl, substituted or unsubstituted C2 to C20 alkenyl, substituted or unsubstituted C2 to C20 alkynyl, substituted or unsubstituted C3 to C20 cycloalkyl, substituted or unsubstituted C3 to C20 cycloalkenyl, substituted or unsubstituted C3 to C20 cycloalkynyl, substituted or unsubstituted C2 to C20 heterocycloalkyl, substituted or unsubstituted C2 to C20 heterocycloalkenyl, substituted or unsubstituted C2 to C20 heterocycloalkynyl, substituted or unsubstituted C6 to C30 aryl, or substituted or unsubstituted C6 to C30 aryloxy.

Examples of the methine-based dye may be represented by Chemical Formula 1 in which R¹ and R² can be the same or different and may independently include substituted or unsubstituted C3 to C20 cycloalkyl, substituted or unsubstituted C3 to C20 cycloalkenyl, substituted or unsubstituted C3 to C20 cycloalkynyl, substituted or unsubstituted C2 to C20 heterocycloalkyl, substituted or unsubstituted C2 to C20 heterocycloalkenyl, substituted or unsubstituted C2 to C20 heterocycloalkynyl, substituted or unsubstituted C6 to C30 aryl, or substituted or unsubstituted C6 to C30 aryloxy.

In an exemplary embodiment, R¹ of the methine-based dye may include hydrogen or substituted or unsubstituted C1 to C20 alkyl and R² may include substituted or unsubstituted phenyl, substituted or unsubstituted aniline, substituted or unsubstituted aniline derivative, substituted or unsubstituted cyclopentyl, substituted or unsubstituted morpholinyl, substituted or unsubstituted naphthyl, substituted or unsubstituted benzyloxy, substituted or unsubstituted pyrimidinyl, substituted or unsubstituted pyridinyl, substituted or unsubstituted quinolinyl, substituted or unsubstituted quinoxalinyl, substituted or unsubstituted pyrazinyl, substituted or unsubstituted pyrazolyl, substituted or unsubstituted benzimidazolyl, substituted or unsubstituted imidazolyl, substituted or unsubstituted dihydrobenzofuranyl, substituted or unsubstituted triazolyl, or substituted or unsubstituted oxazolyl, for example substituted or unsubstituted aniline or substituted or unsubstituted aniline derivative.

In exemplary embodiments, the methine-based dye may be represented by the following Chemical Formula 2.

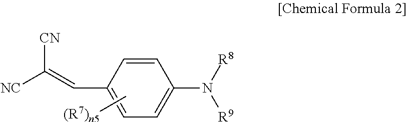

In Chemical Formula 2,

each R⁷ is independently hydrogen, substituted or unsubstituted C1 to C20 alkoxy, substituted or unsubstituted C1 to C20 alkyl, substituted or unsubstituted C6 to C30 aryl, or substituted or unsubstituted C6 to C30 aryloxy,

R⁸ and R⁹ are the same or different and independently are hydrogen, substituted or unsubstituted C1 to C20 alkoxy, substituted or unsubstituted C1 to C20 alkyl, substituted or unsubstituted C6 to C30 aryl, substituted or unsubstituted C6 to C30 aryloxy, or ether, and

n⁵ is an integer ranging from 1 to 4.

The dye may be well-dissolved in an organic solvent and thus may have substantially no graininess or a small primary particle diameter ranging from 1 to 10 nm, unlike a grainy pigment. The dye also can have high durability. When the dye has a smaller particle diameter than a pigment, it may decrease optical scattering and as a result can improve the contrast ratio of a photosensitive resin composition. Accordingly, it can compensate for problems of contrast ratio and luminance deterioration that can be associated with pigment dispersion methods conventionally used to prepare a color filter.

The dye may have a solubility ranging from 3 to 20 in an organic solvent, for example from 3 to 12.

In some embodiments, the methine-based dye represented by Chemical Formula 1 may have a solubility of 3, 4, 5, 6, 7, 8, 9, 10, 11, 12, 13, 14, 15, 16, 17, 18, 19, or 20. Further, according to some embodiments of the present invention, the solubility of the methine-based dye can be in a range from any of the foregoing amounts to any other of the foregoing amounts.

When the dye has a solubility within these ranges, the dye may not be as easily extracted and also can have excellent coloring and compatibility with other components included in a photosensitive resin composition for a color filter.

The organic solvent for determining solubility of the methine-based dye represented by Chemical Formula 1 may be the same as the following solvent (E). In exemplary embodiments, the solvent may include propylene glycol monomethylether acetate (PGMEA), ethyl 3-ethoxypropionate, cyclohexanone, and the like.

The solubility of the methine-based dye may be measured according to the following method.

The dye may be dissolved in 100 g of the solvent and the solution may be agitated for about 5 minutes to obtain a saturated solution at room temperature. 5 ml of the solution at the time when dye precipitation starts to generate may be collected, and then its weight may be measured. Then, the solution may be dried on the oven at about 160° for about 2 hours, and then the weight of the dye (unit: g) may be measured, thereby measuring the solubility of the dye.

The dye may have a maximum absorption wavelength within a wavelength region ranging from 400 to 460 nm, and a transmittance ranging from 95 to 100% within a wavelength region ranging from 500 to 800 nm in spectral analysis. When the dye has a maximum absorption wavelength and/or a transmittance within these ranges, it can provide high luminance.

The dye may have high heat resistance. In other words, it may be thermally decomposed at 200° C. or higher, for example, at a temperature ranging from 200 to 400° C. when measured with a thermogravimetric analyzer (TGA).

The dye having the above characteristics can be useful in the production of various products including without limitation a color filter such as for a LCD, LED, and the like, can provide high luminance and can provide a high contrast ratio in a desired color coordinate.

The photosensitive resin composition for a color filter may further include yellow dyes other than the methine-based dye.

The photosensitive resin composition for a color filter may include the methine-based dye represented by Chemical Formula 1, and when present any additional yellow dye(s) other than the methine-base dye in an amount ranging from 0.1 to 20 wt %, for example, from 5 to 15 wt % based on the entire amount of the photosensitive resin composition for a color filter.

In some embodiments, the photosensitive resin composition for a color filter may include the methine-based dye represented by Chemical Formula 1, and when present any additional yellow dye(s), in an amount of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 wt %. Further, according to some embodiments of the present invention, the amount of the methine-based dye represented by Chemical Formula 1, and when present any additional yellow dye(s), can be in a range from any of the foregoing amounts to any other of the foregoing amounts.

When the yellow dye(s), including the methine-based dye of Chemical Formula 1, is used in an amount within these ranges, it can provide high luminance in the same color coordinate.

(A′) Pigment

The photosensitive resin composition for a color filter may further include a pigment.

The pigment may be red or green.

The red pigment may include at least one azo group represented by the following Chemical Formula 3.

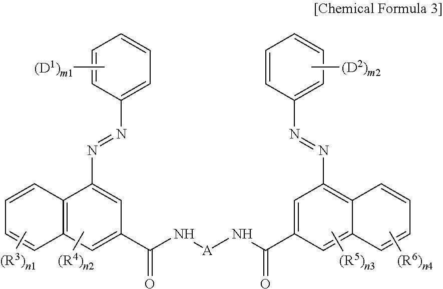

In Chemical Formula 3,

A is substituted or unsubstituted C6 to C30 arylene, substituted or unsubstituted C3 to C30 cycloalkylene, or substituted or unsubstituted C2 to C30 heterocycloalkylene,

each D¹ and D² is the same or different and is independently substituted or unsubstituted C6 to C30 aryl, substituted or unsubstituted C3 to C30 cycloalkyl, or substituted or unsubstituted C2 to C30 heterocycloalkyl,

each R³ to R⁶ is the same or different and is independently hydrogen, hydroxyl, nitro, amine, cyano, carboxylic acid, substituted or unsubstituted C1 to C20 alkyl, or substituted or unsubstituted C6 to C30 aryl,

n¹ to n⁴ are independently integers ranging from 0 to 4, and

m¹ and m² are independently integers ranging from 1 to 5.

In the above Chemical Formula 3, A may be a connecting group represented by one of the following Chemical Formulae 4-1 to 4-3, and each D¹ and D² may be the same or different and may be one of the substituents represented by the following Chemical Formulae 5-1 to 5-6.

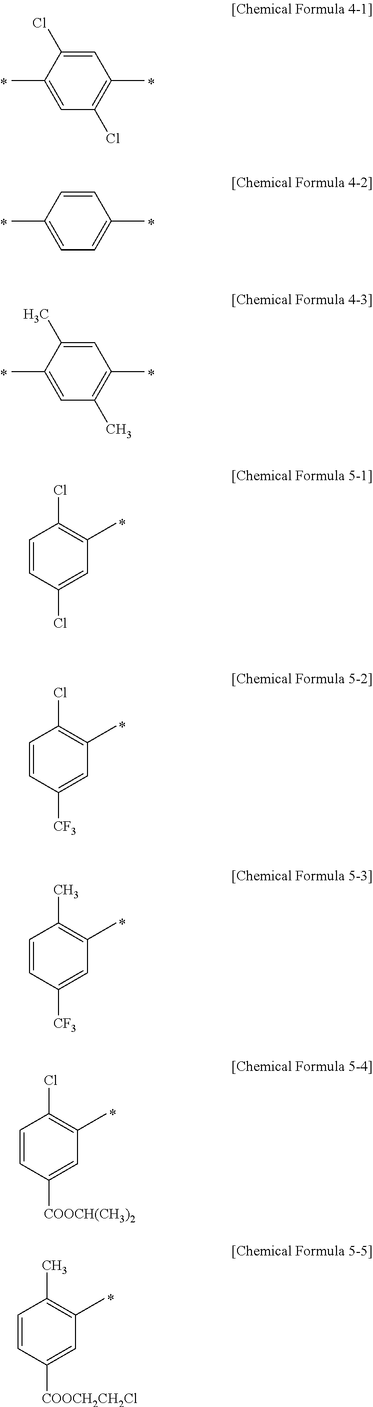

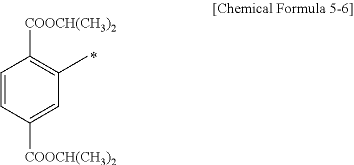

Exemplary red pigments may include without limitation C.I. pigment red 254, C.I. pigment red 242, C.I. pigment red 214, C.I. pigment red 221, C.I. pigment red 166, C.I. pigment red 220, C.I. pigment red 248, C.I. pigment red 262, and the like in a color index. They may be used singularly or as a mixture of two or more.

The main pigment may be mixed with an assistant pigment having excellent light and heat resistance in order to secure color characteristics. The assistant pigment may include C.I. pigment red 177, C.I. pigment yellow 150, and the like, and combinations thereof.

Exemplary green pigments may include without limitation C.I. pigment green 36, C.I. pigment green 58, and the like, and combinations thereof.

The pigment may be prepared as a dispersion solution and included in the photosensitive resin composition for a color filter of the invention. The pigment dispersion solution may include the pigment and a solvent, a dispersing agent, a binder resin, and the like.

Exemplary solvents for the pigment dispersion may include without limitation ethylene glycol acetate, ethyl cellosolve, propylene glycol methyl ether acetate, ethyl lactate, polyethylene glycol, cyclohexanone, propylene glycol methyl ether, and the like, and combinations thereof. In exemplary embodiments, the solvent for the pigment dispersion can be propylene glycol methyl ether acetate.

The dispersing agent helps to uniformly disperse the pigment. Exemplary dispersing agents may include without limitation non-ionic, anionic, or cationic dispersing agents. Examples of the dispersing agent include without limitation polyalkylene glycol and esters thereof, polyoxy alkylene, polyhydric alcohol ester alkylene oxide addition products, alcohol alkylene oxide addition products, sulfonic acid esters, sulfonic acid salts, carboxylic acid esters, carboxylic acid salts, alkyl amide alkylene oxide addition products, alkyl amines, and the like. These dispersing agents can be used singularly or in a combination of two or more.

Examples of the binder resin include a carboxyl-containing acrylic-based resin, and the like. The binder resin can improve the pattern of pixels as well as stability of a pigment dispersion solution.

The pigment can have a primary particle diameter ranging from 10 to 70 nm. When the pigment has a primary particle diameter within the above range, it can have excellent stability in a dispersion solution and may improve pixel resolution.

In addition, there is no particular limit to the secondary particle diameter of the pigment dispersed in the dispersion solution. The pigment dispersed in the pigment dispersion may have a secondary particle diameter of less than 200 nm, taking into account pixel resolution. In another embodiment, the pigment of the pigment dispersion may have a secondary particle diameter ranging from 70 to 100 nm.

The photosensitive resin composition for a color filter may include the pigment in an amount ranging from 25 to 50 parts by weight, for example from 30 to 45 parts by weight, based on 100 parts by weight of the photosensitive resin composition for a color filter. In some embodiments, the photosensitive resin composition for a color filter may include the pigment in an amount of 25, 26, 27, 28, 29, 30, 31, 32, 33, 34, 35, 36, 37, 38, 39, 40, 41, 42, 43, 44, 45, 46, 47, 48, 49, or 50 parts by weight. Further, according to some embodiments of the present invention, the amount of the pigment can be in a range from any of the foregoing amounts to any other of the foregoing amounts.

When the pigment is included in an amount within these ranges, the composition may have excellent coloring and developing properties.

The dye and the pigment can be mixed in a weight ratio ranging from 1:9 to 9:1, for example, from 3:7 to 7:3. When mixed within these ranges, the composition can maintain color characteristics and also can have high luminance and contrast ratio.

(B) Acrylic-Based Binder Resin

The acrylic-based binder resin is a copolymer of a first ethylenic unsaturated monomer and a second ethylenic unsaturated monomer that is copolymerizable with the first ethylenic unsaturated monomer, and a resin including at least one acrylic-based repeating unit.

The first ethylenic unsaturated monomer is an ethylenic unsaturated monomer including at least one carboxyl group. Examples of the first ethylenic unsaturated monomer including at least one carboxyl group include without limitation acrylic acid, methacrylic acid, maleic acid, itaconic acid, fumaric acid, and the like, and combinations thereof.

The acrylic-based resin may include the first ethylenic unsaturated monomer in an amount ranging from 5 to 50 wt %, for example, from 10 to 40 wt %, based on the entire amount of the acrylic-based binder resin. In some embodiments, the acrylic-based resin may include the first ethylenic unsaturated monomer in an amount of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 wt %. Further, according to some embodiments of the present invention, the amount of the first ethylenic unsaturated monomer can be in a range from any of the foregoing amounts to any other of the foregoing amounts.

Examples of the second, ethylenic unsaturated monomer may include without limitation aromatic vinyl compounds such as styrene, α-methylstyrene, vinyltoluene, vinylbenzylmethylether, and the like; unsaturated carboxylic acid ester compounds such as methyl(meth)acrylate, ethyl(meth)acrylate, butyl(meth)acrylate, 2-hydroxyethyl(meth)acrylate, 2-hydroxy butyl(meth)acrylate, benzyl(meth)acrylate, cyclohexyl(meth)acrylate, phenyl(meth)acrylate, and the like; unsaturated carboxylic acid amino alkyl ester compounds such as 2-aminoethyl(meth)acrylate, 2-dimethylaminoethyl(meth)acrylate, and the like; carboxylic acid vinyl ester compounds such as vinyl acetate, vinyl benzoate, and the like; unsaturated carboxylic acid glycidyl ester compounds such as glycidyl(meth)acrylate and the like; vinyl cyanide compounds such as (meth)acrylonitrile and the like; unsaturated amide compounds such as (meth)acrylamide and the like; and the like. The second ethylenic unsaturated monomer may be used singularly or as a mixture of two of more.

Examples of the acrylic-based binder resin may include without limitation a methacrylic acid/benzylmethacrylate copolymer, a methacrylic acid/benzylmethacrylate/styrene copolymer, a methacrylic acid/benzylmethacrylate/2-hydroxyethylmethacrylate copolymer, a methacrylic acid/benzylmethacrylate/styrene/2-hydroxyethylmethacrylate copolymer, and the like. The acrylic-based binder resin may be used singularly or as a mixture of two or more.

The acrylic-based binder resin may have a weight average molecular weight ranging from 3000 to 150,000, for example, from 5000 to 50,000, and as another example, from 20,000 to 30,000. When the acrylic-based binder resin has a weight average molecular weight within these ranges, the composition may have an excellent close contacting (adhesive) property with a substrate, good physical and chemical properties, and appropriate viscosity.

The acrylic-based binder resin may have an acid value ranging from 15 to 60 mgKOH/g, for example, from 20 to 50 mgKOH/g. When the acrylic-based binder resin has an acid value within these ranges, it can bring about excellent pixel resolution.

The photosensitive resin composition for a color filter may include the acrylic-based binder resin in an amount ranging from 1 to 30 wt %, for example, from 5 to 20 wt %, based on the total amount of the photosensitive resin composition for a color filter. In some embodiments, the photosensitive resin composition for a color filter may include the acrylic-based binder resin in an amount of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 wt %. Further, according to some embodiments of the present invention, the amount of the acrylic-based binder resin can be in a range from any of the foregoing amounts to any other of the foregoing amounts.

When the acrylic-based binder resin is included in an amount within these ranges, the composition may have an excellent developing property and improved cross-linking, and thus can have excellent surface flatness when fabricated into a color filter.

(C) Photopolymerization Initiator

The photopolymerization initiator can be any photopolymerization initiator known in art for preparing a photosensitive resin composition for a color filter. Exemplary photopolymerization initiators may include without limitation acetophenone-based compounds, benzophenone-based compounds, thioxanthone-based compounds, benzoin-based compounds, triazine-based compounds, oxime-based compounds, and the like, and combinations thereof.

Exemplary acetophenone-based compounds may include without limitation 2,2′-diethoxyacetophenone, 2,2′-dibutoxyacetophenone, 2-hydroxy-2-methylpropiophenone, p-t-butyltrichloroacetophenone, p-t-butyldichloroacetophenone, 4-chloroacetophenone, 2,2′-dichloro-4-phenoxyacetophenone, 2-methyl-1-(4-(methylthio)phenyl)-2-morpholinopropane-1-one, 2-benzyl-2-dimethylamino-1-(4-morpholinophenyl)-butane-1-one, and the like, and combinations thereof.

Exemplary benzophenone-based compounds may include without limitation benzophenone, benzoyl benzoic acid, benzoyl benzoic acid methyl, 4-phenyl benzophenone, hydroxy benzophenone, acrylic benzophenone, 4,4′-bis(dimethyl amino)benzophenone, 4,4′-bis(diethylamino)benzophenone, 4,4′-dimethylaminobenzophenone, 4,4′-dichlorobenzophenone, 3,3′-dimethyl-2-methoxybenzophenone, and the like, and combinations thereof.

Exemplary thioxanthone-based compounds may include without limitation thioxanthone, 2-methylthioxanthone, isopropyl thioxanthone, 2,4-diethyl thioxanthone, 2,4-diisopropyl thioxanthone, 2-chlorothioxanthone, and the like, and combinations thereof.

Exemplary benzoin-based compounds may include without limitation benzoin, benzoin methyl ether, benzoin ethyl ether, benzoin isopropyl ether, benzoin isobutyl ether, benzyldimethylketal, and the like, and combinations thereof.

Exemplary triazine-based compounds may include without limitation 2,4,6-trichloro-s-triazine, 2-phenyl 4,6-bis(trichloromethyl)-s-triazine, 2-(3′,4′-dimethoxystyryl)-4,6-bis(trichloromethyl)-s-triazine, 2-(4′-methoxynaphthyl)-4,6-bis(trichloromethyl)-s-triazine, 2-(p-methoxyphenyl)-4,6-bis(trichloromethyl)-s-triazine, 2-(p-tolyl)-4,6-bis(trichloro methyl)-s-triazine, 2-biphenyl 4,6-bis(trichloro methyl)-s-triazine, bis(trichloromethyl)-6-styryl-s-triazine, 2-(naphtho-1-yl)-4,6-bis(trichloromethyl)-s-triazine, 2-(4-methoxynaphtho-1-yl)-4,6-bis(trichloromethyl)-s-triazine, 2-4-trichloromethyl(piperonyl)-6-triazine, 2-4-trichloromethyl (4′-methoxystyryl)-6-triazine, and the like, and combinations thereof.

Exemplary oxime-based compounds may include without limitation 2-(o-benzoyloxime)-1-[4-(phenylthio)phenyl]-1,2-octanedione, 1-(o-acetyloxime)-1-[9-ethyl-6-(2-methylbenzoyl)-9H-carbazole-3-yl]ethanone, and the like, and combinations thereof.

Exemplary photopolymerization initiators may also include carbazole-based compounds, diketone-based compounds, sulfonium borate-based compounds, diazo-based compounds, nonimidazole-based compounds, and the like, and combinations thereof, in addition to or as an alternative to the aforementioned photopolymerization initiators.

The photosensitive resin composition for a color filter may include the photopolymerization initiator in an amount ranging from 0.1 to 10 wt %, for example, from 0.5 to 5 wt %, based on the entire amount of the photosensitive resin composition for a color filter. In some embodiments, the photosensitive resin composition for a color filter may include the photopolymerization initiator in an amount of 0.1, 0.2, 0.3, 0.4, 0.5, 0.6, 0.7, 0.8, 0.9, 1, 2, 3, 4, 5, 6, 7, 8, 9, or 10 wt %. Further, according to some embodiments of the present invention, the amount of the photopolymerization initiator can be in a range from any of the foregoing amounts to any other of the foregoing amounts.

When the photopolymerization initiator is included in an amount within these ranges, the composition may be sufficiently photopolymerized when exposed to light during the pattern-forming process for preparing a color filter, and can exhibit excellent sensitivity and improved transmittance.

(D) Photopolymerization Monomer

The photopolymerization monomer may include a multi-functional monomer having two or more hydroxyl groups. Examples of the photopolymerization monomer may include without limitation glycerol acrylate, dipentaerythritol hexaacrylate, ethylene glycol diacrylate, triethylene glycol diacrylate, 1,4-butanedioldiacrylate, 1,6-hexanediol diacrylate, neopentyl glycol diacrylate, pentaerythritol diacrylate, pentaerythritol triacrylate, pentaerythritol diacrylate, dipentaerythritol triacrylate, dipentaerythritol acrylate, pentaerythritol hexaacrylate, bisphenol A diacrylate, trimethylol propane triacrylate, novolacepoxy acrylate, ethylene glycol dimethacrylate, diethylene glycol dimethacrylate, triethylene glycol dimethacrylate, propylene glycol dimethacrylate, 1,4-butanediol dimethacrylate, 1,6-hexanediol dimethacrylate, and the like, and combinations thereof.

The photosensitive resin composition for a color filter may include the photopolymerization monomer in an amount ranging from 1 to 15 wt %, for example, from 5 to 10 wt %, based on the entire amount of the photosensitive resin composition for a color filter. In some embodiments, the photosensitive resin composition for a color filter may include the photopolymerization monomer in an amount of 1, 2, 3, 4, 5, 6, 7, 8, 9, 10, 11, 12, 13, 14, or 15 wt %. Further, according to some embodiments of the present invention, the amount of the photopolymerization monomer can be in a range from any of the foregoing amounts to any other of the foregoing amounts.

When the photopolymerization monomer is included in an amount within these ranges, the composition may have excellent pattern characteristics and developing property.

(E) Solvent

The solvent is not specifically limited. Examples of the solvent include without limitation alcohols such as methanol, ethanol, and the like; ethers such as dichloroethyl ether, n-butyl ether, diisoamyl ether, methylphenyl ether, tetrahydrofuran, and the like; glycol ethers such as ethylene, glycol monomethylether, ethylene glycol monoethylether, and the like; cellosolve acetates such as methyl cellosolve acetate, ethyl cellosolve acetate, diethyl cellosolve acetate, and the like; carbitols such as methylethyl carbitol, diethyl carbitol, diethylene glycol monomethylether, diethylene glycol monoethylether, diethylene glycol dimethylether, diethylene glycol methylethylether, diethylene glycol diethylether, and the like; propylene glycol alkylether acetates such as propylene glycol methylethyl acetate, propylene glycol methylether acetate, propylene glycol propylether acetate, and the like; aromatic hydrocarbons such as toluene, xylene, and the like; ketones such as methylethylketone, cyclohexanone, 4-hydroxy-4-methyl-2-pentanone, methyl-n-propylketone, methyl-n-butylketone, methyl-n-amylketone, 2-heptanone, and the like; saturated aliphatic monocarboxylic acid alkyl esters such as ethyl acetate, n-butyl acetate, isobutyl acetate, and the like; lactic acid alkyl esters such as methyl lactate, ethyl lactate, and the like; hydroxyacetic acid alkyl esters such as methyl hydroxyacetate, ethyl hydroxyacetate, butyl hydroxyacetate, and the like; acetic acid alkoxyalkyl esters such as methoxymethyl acetate, methoxyethyl acetate, methoxybutyl acetate, ethoxymethyl acetate, ethoxyethyl acetate, and the like; 3-hydroxypropionic acid alkyl esters such as methyl 3-hydroxypropionate, ethyl 3-hydroxypropionate, and the like; 3-alkoxypropionic acid alkyl esters such as methyl 3-methoxypropionate, ethyl 3-methoxypropionate, ethyl 3-ethoxypropionate, methyl 3-ethoxypropionate, and the like; 2-hydroxypropionic acid alkyl esters such as methyl 2-hydroxypropionate, ethyl 2-hydroxypropionate, propyl 2-hydroxypropionate, and the like; 2-alkoxypropionic acid alkyl esters such as methyl 2-methoxypropionate, ethyl 2-methoxypropionate, ethyl 2-ethoxypropionate, methyl 2-ethoxypropionate, and the like; 2-oxy-2-methylpropionic acid alkyl esters such as methyl 2-hydroxy-2-methylpropionate, ethyl 2-hydroxy-2-methylpropionate, and the like; 2-alkoxy-2-methylpropionic acid alkyl esters such as methyl 2-methoxy-2-methylpropionate, ethyl 2-ethoxy-2-methylpropionate, and the like; esters such as 2-hydroxyethyl propionate, 2-hydroxy-2-methylethyl propionate, hydroxyethyl acetate, methyl 2-hydroxy-3-methylbutanoate; ketonic acid esters such as ethyl pyruvate; and the like, and combinations thereof. Furthermore, the solvent may be N-methylformamide, N,N-dimethyl formamide, N-methylformanilide, N-methylacetamide, N,N-dimethyl acetamide, N-methylpyrrolidone, dimethylsulfoxide, benzylethylether, dihexylether, acetyl acetone, isophorone, caproic acid, caprylic acid, 1-octanol, 1-nonanol, benzyl alcohol, benzyl acetate, ethyl benzoate, diethyl oxalate, diethyl maleate, γ-butyrolactone, ethylene carbonate, propylene carbonate, phenyl cellosolve acetate, and the like. These solvents may be used singularly or in a combination.

In exemplary embodiments, taking into account factors such as miscibility, reactivity, and the like, examples of the solvent may include without limitation glycol ethers such as ethylene glycol monoethyl ether and the like; ethylene glycol alkylether acetates such as ethyl cellosolve acetate and the like; esters such as 2-hydroxyethyl propionate and the like; diethylene glycols such as diethylene glycol monomethyl ether and the like; propylene glycol alkylether acetates such as propylene glycol monomethylether acetate, propylene glycol propylether acetate, and the like; and combinations thereof.

The solvent may be included in a balance amount, for example, in an amount ranging from 20 to 90 wt %, based on the entire amount of the photosensitive resin composition for a color filter. In some embodiments, the photosensitive resin composition for a color filter may include the solvent in an amount of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 wt %. Further, according to some embodiments of the present invention, the amount of the solvent can be in a range from any of the foregoing amounts to any other of the foregoing amounts.

When the solvent is included in an amount within these ranges, the photosensitive resin composition for a color filter may have excellent coating properties and can maintain excellent flatness in a layer having a thickness of 3 μm or more.

(F) Surfactant

The photosensitive resin composition for a color filter may further include a surfactant to uniformly disperse the pigment into the solvent and to improve leveling performance.

The surfactant may be fluorine-based.

The fluorine-based surfactant may have a weight average molecular weight ranging from 4000 to 10,000, for example, from 6000 to 10,000. In addition, the fluorine-based surfactant may have surface tension ranging from 18 to 23 mN/m (measured in a 0.1% polyethylene glycol methylether acrylate (PEGMEA) solution). When the fluorine-based surfactant has a weight average molecular weight and surface tension within these ranges, the composition can have improved leveling performance and an excellent stain characteristic during the high speed coating. In addition, the composition may generate less vapor and less defects in a layer, so that the composition can be good for a slit coating method, which is a high speed coating method.

Examples of the fluorine-based surfactant may include without limitation F-482, F-484, F-478, and the like, and combinations thereof, which are commercially available from DIC Co., Ltd.

Other exemplary surfactants may include silicon-based surfactants, in addition to or as an alternative to the fluorine-based surfactant.

Examples of the silicon-based surfactant may include without limitation TSF400, TSF401, TSF410, TSF4440, and the like, and combinations thereof, which are commercially available from Toshiba Silicon Co., Ltd.

The photosensitive resin composition for a color filter may include the surfactant in an amount ranging from 0.01 to 5 parts by weight, for example, from 0.1 to 2 parts by weight, based on 100 parts by weight of the photosensitive resin composition for a color filter. When the surfactant is included in an amount within these ranges, the composition may have fewer impurities generated after development.

(G) Other Additives

The photosensitive resin composition for a color filter may further include other additives such as but not limited to malonic acid, 3-amino-1,2-propanediol, and/or a vinyl- or (meth)acryloxy-containing silane-based coupling agent, in order to prevent stains or spots during the coating, to adjust leveling, or to prevent pattern residue due to non-development. These additives may be added in conventional amounts, which can be readily selected by the skilled artisan based on desired properties.

In addition, the photosensitive resin composition for a color filter may additionally include an epoxy compound to improve the close contacting (adhesive) property and other characteristics if needed.

Exemplary epoxy compounds may include without limitation epoxy novolac acryl carboxylate resins, ortho cresol novolac epoxy resins, phenol novolac epoxy resins, tetra methyl biphenyl epoxy resins, bisphenol A-type epoxy resins, alicyclic epoxy resins, and the like, and combinations thereof.

When the epoxy compound is included, a peroxide initiator or a radical polymerization initiator such as an azobis-based initiator can be additionally included.

The photosensitive resin composition for a color filter may include the epoxy compound in an amount of 0.01 to 5 parts by weight, based on 100 parts by weight of the photosensitive resin composition for a color filter. When the epoxy compound is included in an amount ranging from 0.01 to 5 parts by weight, it can improve storage, close contacting (adhesion), and other properties.

There is no particular limitation regarding the method used to prepare the photosensitive resin composition for a color filter. The photosensitive resin composition for a color filter can be prepared by mixing the aforementioned dye, acrylic-based binder resin, photopolymerization initiator, photopolymerization Monomer, and solvent, and optionally one or more additives.

According to another embodiment, provided is a color filter fabricated using the photosensitive resin composition for a color filter.

The color filter may be fabricated using any suitable conventional method. For example, the color filter may be prepared using spin-coating, roller-coating, slit-coating, and the like to form a layer of the composition on a suitable substrate, the layer having a thickness ranging from 1.5 to 2.0 μm. After coating the composition to form a layer, the layer is exposed to radiation, such as UV radiation, electron beam, or X-ray to form a pattern required for a color filter. The UV radiation may have a wavelength region ranging from 190 to 450 nm, for example, from 200 to 400 nm. Next, the coated layer is treated with an alkali developing solution, and the unradiated (non-exposed) region thereof may be dissolved, forming a pattern for an image color filter. This process can be repeated as necessary to form the desired number of R, G, and B colors, to fabricate a color filter having a desired pattern. In addition, the image pattern acquired by the development step can be cured through heat treatment, actinic ray radiation, or the like, which can improve crack resistance, solvent resistance, and the like.

Hereinafter, the present invention is illustrated in more detail with reference to examples. However, these are exemplary embodiments of present invention and are not limiting.

Preparation of a Photosensitive Resin Composition for a Color Filter

A photosensitive resin composition for a color filter may include a component as follows.

(A) Dye

(A-1) A methine-based yellow dye represented by the following Chemical Formula 6 (CF Y100103, KISCO (Korea) Co., Ltd.), which has solubility of 7 in propylene glycol monomethylether acetate (PGMEA) is used.

(A-2) A methine-based yellow dye represented by the following Chemical Formula 7 (CF Y100107, KISCO (Korea) Co., Ltd.), which has solubility of 5 in propylene glycol monomethylether acetate (PGMEA) is used.

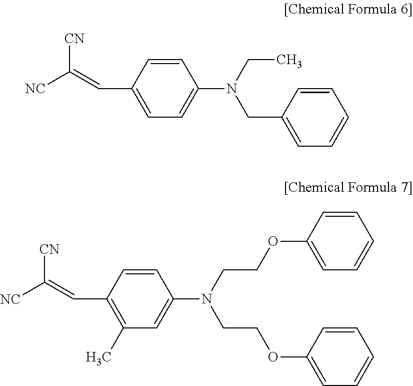

(A′) Pigment

(A′-1) A mixture of C.I. pigment red 254 and C.I. pigment red 177 mixed in a weight ratio of 60:40 is used.

(A′-2) A C.I. pigment yellow 150 is used.

(B) Acrylic-Based Binder Resin

A methacrylic acid/benzylmethacrylate copolymer having a weight average molecular weight of 28,000, which is mixed in a weight ratio of 30:70, is used.

(C) Photopolymerization Initiator

CGI-124 made by Ciba Specialty Chemicals Co is used.

(D) Photopolymerization Monomer

Dipentaerythritolhexaacrylate is used.

(E) Solvent

Propylene glycol monomethyl ether acetate and ethyl 3-ethoxypropionate are used.

(F) Surfactant

A fluorine-based surfactant (F-482, DIC Co., Ltd.) is used.

### Example 1

1.7 g of the photopolymerization initiator (C) is dissolved in 31.1 g of propylene glycol monomethyl ether acetate and 17.3 g of ethyl 3-ethoxypropionate as the solvent (E). The solution is agitated for 2 hours at room temperature. Next, 3.9 g of a yellow dye (A-1) represented by the above Chemical Formula 6, 3.5 g of the acrylic-based binder resin (B), and 8.5 g of the photopolymerization monomer (D) are added thereto. The mixture is agitated for 2 hours at room temperature. Then, 33.8 g of the pigment (A′-1) is added thereto. The resulting mixture is agitated for 1 hour at room temperature. Then, 0.2 g of the surfactant (F) is added thereto. The mixture is agitated for 1 hour at room temperature. The solution is filtrated three times to remove impurities, preparing a photosensitive resin composition for a color filter.

### Example 2

A photosensitive resin composition for a color filter is prepared according to the same method as Example 1, except for using 3.9 g of a yellow dye (A-2) represented by the above Chemical Formula 7 instead of 3.9 g of the dye (A-1).

### Comparative Example 1

A photosensitive resin composition for a color filter is prepared according to the same method as Example 1, except for using 3.9 g of the pigment (A′-2) instead of the dye (A-1).

### Example 3

1.7 g of the photopolymerization initiator (C) is dissolved in 31.1 g of propylene glycol monomethyl ether acetate and 17.3 g of ethyl 3-ethoxypropionate as the solvent (E). The solution is agitated for 2 hours at room temperature. Next, 1.2 g of a yellow dye (A-1) represented by the above Chemical Formula 6, 3.5 g of the acrylic-based binder resin (B), and 8.5 g of the photopolymerization monomer (D) are added thereto. The mixture is agitated for 2 hours at room temperature. Then, 33.8 g of the pigment (A′-1) and 2.7 g of the pigment (A′-2) are added thereto. The resulting product is agitated for one hour at room temperature. Then, 0.2 g of the surfactant (F) is added thereto and agitated for one hour at room temperature. The solution is filtrated three times to remove impurities, preparing a photosensitive resin composition for a color filter.

### Example 4

A photosensitive resin composition for a color filter is prepared according to the same method as Example 3, except for using the dye (A-1) in an amount of 1.9 g instead of 1.2 g and the pigment (A′-2) in an amount of 2.0 g instead of 2.7 g.

### Example 5

A photosensitive resin composition for a color filter is prepared according to the same method as Example 3, except for using the dye (A-1) in an amount of 2.7 g instead of 1.2 g and the pigment (A′-2) in an amount of 1.2 g instead of 2.7 g.

### Example 6

A photosensitive resin composition for a color filter is prepared according to the same method as Example 3, except for using the dye (A-1) in an amount of 3.9 g instead of 1.2 g and no pigment (A′-2).

### Example 7

1.7 g of the photopolymerization initiator (C) is dissolved in 31.1 g of the propylene glycol monomethyl ether acetate and 17.3 g of ethyl 3-ethoxypropionate as the solvent (E). The solution is agitated for 2 hours at room temperature. Next, 1.2 g of a yellow dye (A-2) represented by the above Chemical Formula 7, 3.5 g of the acrylic-based binder resin (B), and 8.5 g of the photopolymerization monomer (D) are added thereto. The resulting mixture is agitated for 2 hours at room temperature. Next, 33.8 g of the pigment (A′-1) and 2.7 g of the pigment (A′-2) are added thereto. The mixture is agitated for 1 hour at room temperature. Then, 0.2 g of the surfactant (F) is added thereto and agitated for one hour at room temperature. The solution is filtrated three times to remove impurities, preparing a photosensitive resin composition for a color filter.

### Example 8

A photosensitive resin composition for a color filter is prepared according to the same method as Example 7, except for using the dye (A-2) in an amount of 1.9 g instead of 1.2 g and the pigment (A′-2) in an amount of 2.0 g instead of 2.7 g.

### Example 9

A photosensitive resin composition for a color filter is prepared according to the same method as Example 7, except for using the dye (A-2) in an amount of 2.7 g instead of 1.2 g and the pigment (A′-2) in an amount of 1.2 g instead of 2.7 g.

### Example 10

A photosensitive resin composition for a color filter is prepared according to the same method as Example 7, except for using the dye (A-2) in an amount of 3.9 g instead of 1.2 g and no pigment (A′-2).

### Comparative Example 2

A photosensitive resin composition for a color filter is prepared according to the same method as Example 7, except for using no dye (A-2) but the pigment (A′-2) in an amount of 3.9 g.

### Experimental Example 1

### Spectral Transmittance Measurement of a Dye

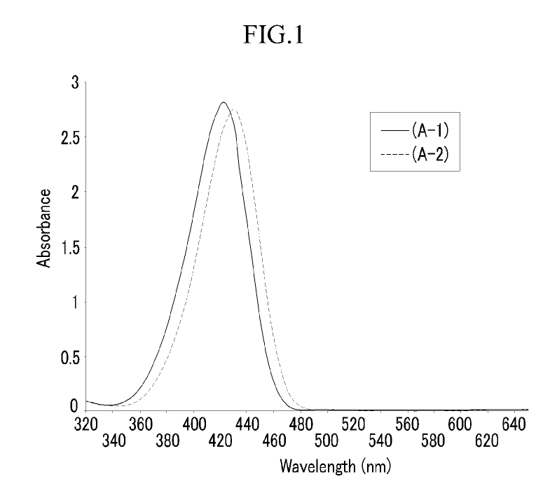

FIG. 1 shows a spectral absorbance graph of methine-based dyes (A-1) according to Examples 1 to 6 and (A-2) Examples 7 to 10.

In order to measure absorbance and transmittance, a dye is diluted 100 times in propylene glycol monomethyl ether acetate. Then, a UV/vis spectrophotometer made by Shimadzu Co. is used.

Referring to FIG. 1, the methine-based yellow dyes (A-1) and (A-2) according to one embodiment are identified to have a maximum absorption wavelength within a wavelength region ranging from 400 to 460 nm, and also a high transmittance ranging from 95 to 100% within a wavelength region ranging from 500 to 800 nm.

Pattern Formation for a Color Filter

The photosensitive resin compositions for a color filter according to Examples 1 to 10 and Comparative Examples 1 and 2, respectively, are coated on a 10×10 cm² glass substrate in a spin-coating method and then pre-baked at 90° C. for 3 minutes. The resulting product is cooled under air and then radiated by UV having a wavelength of 365 nm to an exposure amount of 100 mJ/cm², fabricating a thin film. After the radiation, the thin film is post-baked at 230° C. in a hot air drier for 30 minutes, preparing a cured thin film.

### Experimental Example 2

### Spectral Characteristic Evaluation of a Photosensitive Resin Composition for a Color Filter

The color coordinate, luminance, and contrast ratio of the cured thin films of Examples 1 to 10 and Comparative Examples 1 and 2 are measured according to the following methods. The results are provided in the following Table 1.

(1) Color coordinates (x and y) and luminance (Y): a colorimeter (MCPD 3000, Korea Otsuka Pharmaceutical Co., Ltd.) is used.

(2) Contrast ratio: a contrast ratio measurement device (Tsubosaka Electronic Co. Ltd., CT-1, 20, 000:1) is used.

TABLE 1

| | Color coordinate | | Luminance | |
|---|---|---|---|---|
| | x | y | Y | Contrast ratio |
| Example 1 | 0.651 | 0.330 | 19.4 | 12,800 |
| Example 2 | 0.651 | 0.330 | 19.3 | 12,700 |
| Comparative | 0.651 | 0.330 | 18.5 | 11,500 |
| Example 1 | | | | |
| Example 3 | 0.651 | 0.330 | 18.7 | 11,300 |
| Example 4 | 0.651 | 0.330 | 18.9 | 12,000 |
| Example 5 | 0.651 | 0.330 | 19.1 | 12,300 |
| Example 6 | 0.651 | 0.330 | 19.3 | 12,600 |
| Example 7 | 0.651 | 0.330 | 18.6 | 11,300 |
| Example 8 | 0.651 | 0.330 | 18.8 | 11,900 |
| Example 9 | 0.651 | 0.330 | 19.0 | 12,100 |
| Example 10 | 0.651 | 0.330 | 19.1 | 12,500 |
| Comparative | 0.651 | 0.330 | 18.4 | 11,200 |
| Example 2 | | | | |

Referring to Table 1, Examples 1 and 2, which include a methine-based dye according to exemplary embodiments of the invention, have higher luminance and contrast ratio than Comparative Example 1, which includes a pigment instead of the methine-based dye.

In addition, Examples 3 to 10, which include a methine-based dye according to exemplary embodiments of the invention, have higher luminance and contrast ratio than Comparative Example 2, which includes a pigment instead of the methine-based dye.

Although not wishing to be bound by any explanation or theory of the invention, it is currently believed that the high luminance is the result of high transmittance of the dye. The high contrast ratio is believed to be the result of the dye being soluble in an organic solvent unlike dispersive particles of a pigment, so that the composition has minimal or no graininess or has a significantly smaller primary particle diameter than a pigment, which can decrease unnecessary optical scattering in the photosensitive resin composition for a color filter.

In addition, as shown in Examples 3 to 6 and Examples 7 to 10, as the amount of dye increased, the luminance and contrast ratio increased.

### Experimental Example 3

### Durability Evaluation of a Photosensitive Resin Composition for a Color Filter

The durability of the cured thin films according to Examples 1 and 2 and Comparative Example 1 is measured using the following method. The results are provided in the following Table 2.

The durability is evaluated by dipping the cured thin films (A) in a N-methylpyrrolidone (NMP) solvent at room temperature for 30 minutes, and (B) in a mixed solvent of N-methylpyrrolidone (NMP) and ethylethoxypropionate (hereinafter referred to as “EEP”) mixed in a volume ratio of 5:5 at 80° C. for 10 minutes. ΔEab* is obtained by calculating a color difference before and after treating each cured thin film sample. When a cured thin film sample has ΔEab* of 3 or less, the value is reliable. The smaller the ΔEab* is, the better the durability of the cured thin film.

TABLE 2

ΔEab*

| | (A) NMP | (B) NMP + EEP |
|---|---|---|
| Example 1 | 1.05 | 0.87 |
| Example 2 | 1.30 | 0.63 |
| Comparative Example 1 | 1.50 | 1.03 |

Examples 1 and 2 and Comparative Example 1 all have excellent durability. However, Examples 1 and 2 including a methine-based dye have a lower ΔEab* than Comparative Example 1, which includes a pigment instead of the methine-based dye.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation, the scope of the invention being defined in the claims.

What is claimed is:

1. A photosensitive resin composition for a color filter comprising a methine-based dye represented by the following Chemical Formula 1: 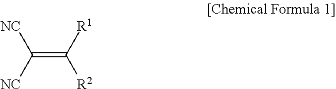
wherein, in Chemical Formula 1,
R¹ and R² are the same or different and independently comprise hydrogen, halogen, hydroxy, ether, amine, amine derivative, substituted or unsubstituted C1 to C20 alkoxy, substituted or unsubstituted C1 to C20 alkyl, substituted or unsubstituted C2 to C20 alkenyl, substituted or unsubstituted C2 to C20 alkynyl, substituted or unsubstituted C3 to C20 cycloalkyl, substituted or unsubstituted C3 to C20 cycloalkenyl, substituted or unsubstituted C3 to C20 cycloalkynyl, substituted or unsubstituted C2 to C20 heterocycloalkyl, substituted or unsubstituted C2 to C20 heterocycloalkenyl, substituted or unsubstituted C2 to C20 heterocycloalkynyl, substituted or unsubstituted C6 to C30 aryl, or substituted or unsubstituted C6 to C30 aryloxy.

2. The photosensitive resin composition of claim 1, wherein the dye has a solubility ranging from 3 to 20 in an organic solvent.

3. The photosensitive resin composition of claim 1, wherein the dye has a maximum absorption wavelength in a wavelength region ranging from 400 to 460 nm.

4. The photosensitive resin composition of claim 1, wherein the dye has a transmittance ranging from 95 to 100% in a wavelength region ranging from 500 to 800 nm.

5. The photosensitive resin composition of claim 1, wherein the dye is thermally decomposed at a temperature ranging from 200 to 400° C.

6. The photosensitive resin composition of claim 1, wherein R¹ and R² independently comprise substituted or unsubstituted C3 to C20 cycloalkyl, substituted or unsubstituted C3 to C20 cycloalkenyl, substituted or unsubstituted C3 to C20 cycloalkynyl, substituted or unsubstituted C2 to C20 heterocycloalkyl, substituted or unsubstituted C2 to C20 heterocycloalkenyl, substituted or unsubstituted C2 to C20 heterocycloalkynyl, substituted or unsubstituted C6 to C30 aryl, or substituted or unsubstituted C6 to C30 aryloxy.

7. The photosensitive resin composition of claim 1, wherein R¹ comprises hydrogen or substituted or unsubstituted C1 to C20 alkyl and R² comprises substituted or unsubstituted phenyl, substituted or unsubstituted aniline, substituted or unsubstituted aniline derivative, substituted or unsubstituted cyclopentyl, substituted or unsubstituted morpholinyl, substituted or unsubstituted naphthyl, substituted or unsubstituted benzyloxy, substituted or unsubstituted pyrimidinyl, substituted or unsubstituted pyridinyl, substituted or unsubstituted quinolinyl, substituted or unsubstituted quinoxalinyl, substituted or unsubstituted pyrazinyl, substituted or unsubstituted pyrazolyl, substituted or unsubstituted benzimidazolyl, substituted or unsubstituted imidazolyl, substituted or unsubstituted dihydrobenzofuranyl, substituted or unsubstituted triazolyl, or substituted or unsubstituted oxazolyl.

8. The photosensitive resin composition of claim 1, wherein the methine-based dye is represented by the following Chemical Formula 2: 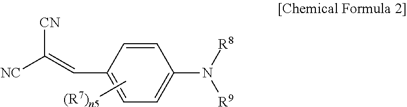
wherein, in Chemical Formula 2,
each R⁷ is independently hydrogen, substituted or unsubstituted C1 to C20 alkoxy, substituted or unsubstituted C1 to C20 alkyl, substituted or unsubstituted C6 to C30 aryl, or substituted or unsubstituted C6 to C30 aryloxy,
R⁸ and R⁹ are the same or different and independently are hydrogen, substituted or unsubstituted C1 to C20 alkoxy, substituted or unsubstituted C1 to C20 alkyl, substituted or unsubstituted C6 to C30 aryl, substituted or unsubstituted C6 to C30 aryloxy, or ether, and
n⁵ is an integer ranging from 1 to 4.

9. The photosensitive resin composition of claim 8, wherein the methine-based dye is a compound of Chemical Formula 6:

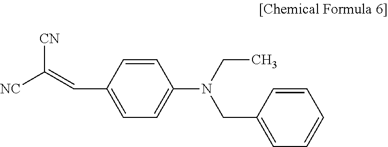

10. The photosensitive resin composition of claim 8, wherein the methine-based dye is a compound of Chemical Formula 7:

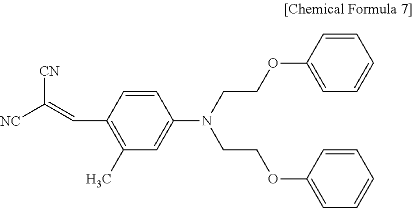

11. The photosensitive resin composition of claim 1, comprising the dye in an amount ranging from 0.1 to 20 wt % based on the entire amount of the photosensitive resin composition for a color filter.

12. The photosensitive resin composition of claim 1, further comprising a pigment.

13. The photosensitive resin composition of claim 12, wherein the pigment is red or green.

14. The photosensitive resin composition of claim 12, wherein the dye and the pigment are mixed in a weight ratio ranging from 1:9 to 9:1.

15. The photosensitive resin composition of claim 1, further comprising an acrylic-based binder resin, a photopolymerization initiator, a photopolymerization monomer, and a solvent.

16. The photosensitive resin composition of claim 15, comprising:
(A) 0.1 to 20 wt % of the methine-based dye represented by the above Chemical Formula 1;
(B) 1 to 30 wt % of the acrylic-based binder resin;
(C) 0.1 to 10 wt % of the photopolymerization initiator;
(D) 1 to 15 wt % of the photopolymerization monomer; and
(E) the solvent in a balance amount.

17. The photosensitive resin composition of claim 1, further comprising a surfactant.

18. A color filter fabricated by using the photosensitive resin composition of claim 1.
